(19) United States
(12) Patent Application Publication
(10) Pub. No.: US 2016/0220197 A1
(43) Pub. Date: Aug. 4, 2016

(54) ALARM GENERATION METHOD AND ARTEFACT REJECTION FOR PATIENT MONITOR

(71) Applicant: Börje Rantala, Helsinki (FI)

(72) Inventor: Börje Rantala, Helsinki (FI)

(21) Appl. No.: 14/960,468

(22) Filed: Dec. 7, 2015

Related U.S. Application Data
(60) Provisional application No. 62/109,116, filed on Jan. 29, 2015.

(51) Int. Cl. A61B 5/00; A61B 5/145; A61B 5/0472; A61B 5/021; A61B 5/046; A61B 5/0476; A61B 5/01; A61B 5/08; A61B 5/0464

(52) U.S. Cl. CPC A61B 5/746; A61B 5/0826; A61B 5/14542; A61B 5/0472; A61B 5/0464; A61B 5/046; A61B 5/14546; A61B 5/0476; A61B 5/01; A61B 5/021; A61B 5/4818

(57) ABSTRACT

A method for managing alarms in a physiological monitor by identifying a clinical sequence of adverse effects and following the evolution of the sequence by alarm logic. By requiring the evolution to proceed as defined, false alarms from the individual parameters can be suppressed, resulting in a significant improvement in the specificity of the alarm, without sacrificing sensitivity.

### BACKGROUND OF THE INVENTION

This disclosure relates generally to patient monitoring. Particularly to alarm generation and accompanying artefact and false alarm rejection.

When a patient is being monitored by with multiple parameters, like respiration (Resp), oxygen saturation (SpO2) and ECG (often ST-segment depression/elevation), the combined parameter set usually generates frequent false alarm from the monitored parameters individually, due to e.g. patient motion. Especially Resp is prone to generate false alarms whether it is derived by a bed or mattress motion sensor or through an impedance measurement from the ECG electrodes. A patient may move off the mattress sensor or breathe with his belly (with electrodes on the chest).

Multiple mechanisms to decrease the number of false alarms have been proposed, many trading off sensitivity to specificity. [3] and [4] are examples of the extensive prior art on the apnea and SpO2 monitoring technologies. [6] describes combining information from motion data and SpO2,

### BRIEF DESCRIPTION OF THE INVENTION

The present invention is a novel approach in reducing false alarms without unduly compromising or trading off sensitivity for specificity. It is based on realizing that in many monitoring situations there is a typical evolution of a dangerous sequence of events. Thus an initial patient adverse state leads to one or more, progressively more dangerous states, enabling a logic following this evolution to be specific in generating the alarm, without alarming on individual patient adverse states of the evolution.

One preferred embodiment of this alarm logic is the evolution of sleep apnea into cardiac failure. A patient, often obese and intoxicated, is sleeping. Heavy snoring evolves into apneic episodes as the patient stops breathing for tens of seconds. As the apneic episodes grow longer, the blood oxygen saturation starts to show drops from its normal values (SpO2 drops under 90%). As the dropping SpO2 reaches critical range (typ. <80%, patient dependent). The cardiac muscle starts suffering from oxygen deprivation which frequently is seen as an ST-segment change, or as arrhythmias. Severe arrhythmias relevant here are e.g. Ventricular Tachycardia (VT), Ventricular Fibrillation (VFib) and Asystole. Other arrhythmias like Atrial Fibrillation can also be classified as severe for most patients.

Taken separately the three individual patient adverse states are prone to artefacts due to patient movements. Thus traditional sleep apnea monitors are plagued by false alarms to the extent that the alarming function often is suppressed. Making the alarm generation (e.g. priority escalation) dependent of the event sequence reduces the number of false alarms compared with alarming on limit violation of the individual parameters.

Of course the individual parameters can be made to alarm individually, but for the specific patient group these alarms can be set to be quite insensitive in order to keep the false alarm rate low.

The set of alarming parameters and events can obviously belong to other physiologic state evolutions without limiting the applicability of the present invention.

### DESCRIPTION OF THE DRAWINGS

FIG. 1 illustrates one embodiment the invention; Here the patient is undergoing sleep apnea monitoring multiple sensors 10 connected: A bed sensor 100, typically a piezoelectric sensor under the mattress, a telemetric wrist sensor for pulse oximetry 200 and a telemetric ECG monitor 300 (often including impedance respiration, with secondary sensing of cardiac motion and patient movements). This embodiment having a combination of non-touch sensors and wireless sensors allows the patient to move and e.g. use the toilet. The signals are sent to a Monitor unit 11 that handles the Viewing and Alarm generation. The algorithms associated with extracting the vital parameters Respiration Rate and Apnea 101, SpO2 201 and ST-Segment/Heat Rate/Arrhythmia/QT-time interval 301, can be implemented either in the Sensors 10 or in the Monitor 11.

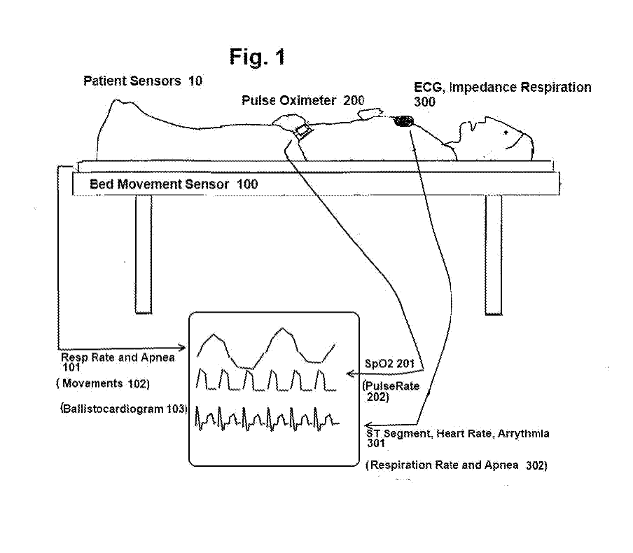

Note that similar parameters, e.g. Respiration Rate 101 from the bed sensor and Respiration Rate 302 from the chest impedance sensor can be used by the algorithms, either together or alternatively, should the other sensor fail or contain noise. The definition of Apnea should here be understood in the wider sense of severe respiratory insufficiency like very shallow breathing, or very low respiratory rate.

FIG. 2 is an example of an apneic sequence. The motion artefacts, finger twisting and electrical noise artefacts would trigger alarms for the individual parameters (401, 402, 403). In FIG. 2 the alarm of the present invention would sound only at the end (404), after the sequence: apnea>120 s→SpO2<85% for 30 s→ST segment<3 mm for 60 s. The individual alarms caused by the noise and the artefacts would be suppressed. Suppressed would also be real physiological changes of short duration unless the limit violations would be large enough to trigger the individual parameter alarm criteria. This is usually acceptable, as the small degree of violation and the short duration are not significant enough to alarm on.

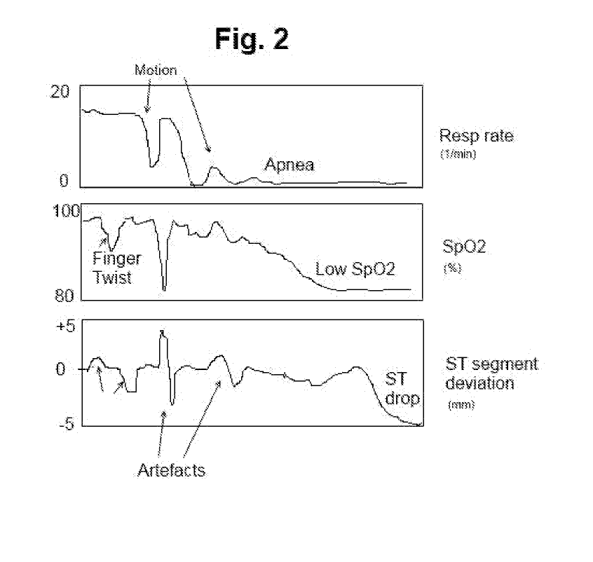

### DETAILS OF THE INVENTION

The sequence of events to generate an alarm is defined as a combination of the subalarms of the individual parameters. Subalarm here means a triggered individual alarm that is hidden, and only used as a part of the logic to generate the final alarm. The subalarms usually have their individual limits and priority escalation rules (ref 1). Priorities are low→medium→high, here represented by 1, 2, 3. The final alarm can then be calculated e.g. as

Priority(Final)=(Priority(1)+A*Alarmtime(1)*Priority(2)+B*Alarmtime(2)*Priority(3)) *normalizing factor,

where A and B depend on the parameters and Alarmtime( ) is the time the previous parameter in the sequence has been subalarming. The normalizing factor brings the final alarm priority into the normal range 1 . . . 3. The alarms (not subalarms, but traditional individual alarms) for the individual parameters (e.g. Respiration rate, SpO2 and ST segment) can be set to much more insensitive setting so as again to avoid false alarms, by e.g. using wider limits or longer alarm activation and escalation delays. The term “subalarm logic” is defined here to include alarm limits, activation delay, alarm priority escalation rules and other rules described in [1].

Typically for these sleep apnea traditional individual alarm settings would be as follows: The priority escalation for apnea only would be priority 1 (“note”) reached after 20 s of apnea, with 60 s resulting in priority 2 (“warning”). The maximum priority for apnea could also be “1” to eliminate alarms due to the patient having moved to a position where the respiration sensor would not function.

The individual priority escalation for SpO2 would be more complex, depending both on the time and extent of the limit violation, such that e.g. SpO2<75% would trigger an immediate priority 3 alarm. According to the new logic a high priority could then be reached as SpO2<85% for 2 min following an apnea sustained for >30 s. The ST segment deviation would alarm individually when being <−4 or >+4 mm for 10 min with low priority (“1”), but following an SpO2 low alarm the priority could reach 2 after ST<−2 or >+2 mm for 5 min.

The alarm function for sleep apnea is both to arouse the patient and to alert family or clinicians. A specialized alarm version would be just an indicator in a Holter recording, directing the reviewing clinician's attention to the relevant part of the recording. This saves clinician time and reduces the probability of missing significant events.

The concept of a predefined sequence of subalarms can also be used to set subalarm logic parameters for one step from the values of the previous parameter in the sequence before it subalarmed. Thus the system would detect changes in the patient state from the pre-alarm state. Among these are automatic setting of subalarm limits in sequence; the SpO2 limit may be the pre-apnea SpO2 averaged over e.g. 1 minute minus 5% SpO2. Similarly the ST baseline could be the pre-apnea 5 minute average, and deviations from this value would be subalarmed on.

The details of the escalation of the final alarm is only indicated here; there are obviously several ways of combining the chain of subalarms or events (factors or statuses affecting the alarm logic without being alarms themselves, e.g. “noise”, or “audio pause”—activated) the to produce the final alarm (“sequence logic”).

Examples of sequential adverse events covered by the present invention are:
LowRespRate→LowSpO2→ST_SegmentDeviation.
LowRespRate→LowSpO2→Arrhythmia.
HighTemperature→LowBloodPressure→Sepsis.
HighTemperature→LowBloodPressure→LowSpO2→Adult Respiratory Distress Syndrome.
   LowHeartRate→LowBloodPressure→Arrhythmia.

### REFERENCES

[1] Alarm generation method for patient monitoring, physiological monitoring apparatus and computer program product for a physiological monitoring apparatus.
U.S. Pat. No. 8,456,295 B2 Börje Rantala 26 May 2010
Original Assignee General Electric Company

[2] Method, Device and Computer Program Product for Monitoring Patients Receiving Care.
US 20120053422 A1 Börje Rantala 24 Aug. 2010
Original Assignee General Electric Company

[3] Body-worn system for continuously monitoring a patient's bp, hr, spo2, rr, temperature, and motion; also describes specific monitors for apnea, asy, vtac, vfib, and ‘bed sore’ index.
US20100298659 A1, Devin McCOMBIE 20 May 2009.

[4] System and method for SPO2 instability detection and quantification
U.S. Pat. No. 8,666,467 B2, Lynn 17 May 2001.

[5] Alarm system that processes both motion and vital signs using specific heuristic rules and thresholds.
U.S. Pat. No. 8,594,776 B2, Devin McCOMBIE 20 May 2009

[6] Alarm system that processes both motion and vital signs using specific heuristic rules and thresholds.
U.S. Pat. No. 8,180,440 B2, Devin McCOMBIE 20 May 2009

[7] Lindberg et. al., “Evolution of Sleep Apnea Syndrome in Sleepy Snorers”, American Journal of Respiratory and Critical Care Medicine, Vol. 159, No. 6 (1999), pp. 2024-2027.

1. A method for generating alarms in a physiological monitor comprising:
Monitoring 2 or more physiological parameters;
Defining a clinical sequence of patient adverse states leading to a final adverse patient state;
Assigning subalarm criteria to the adverse states in the clinical sequence;
Defining sequence logic for a combination of the subalarms, and
Generating a final alarm when the subalarms trigger according to the sequence logic.

2. The method according to claim 1, where the sequence logic includes one or more of the subalarm properties: priority, duration, timing, sensor status.

3. The method according to claim 2, where the sequence logic requires each subalarm to persist for a predetermined duration in order for the next subalarm in the sequence to be activated.

4. The method according to claim 1, wherein the assigning subalarm criteria includes selecting alarm limits from parameter averages prior to one or more earlier subalarms.

5. The method according to claim 2, wherein the defining includes defining a formula for constructing the final alarm as a combination of the alarms in the individual patient adverse states sequential chain.

6. The method according to claims 1-5, wherein the parameters are Respiration (including motion), SpO2 and ECG, and the adverse states Apnea, Low SpO2 and ST segment deviation

7. The method according to claims 1-5, wherein the parameters are Respiration (including motion), SpO2 and ECG, and the adverse states Apnea, Low SpO2 and Severe arrhythmia.

8. The method according to claims 1-5, wherein the parameters are Anesthetic agent concentration, EEG depression and Severe arrhythmia.

9. The method according to claims 1-5, wherein the parameters are HighTemperature, LowBloodPressure and Sepsis.

10. The method according to claims 1-5, wherein the alarm is an event flagged in the patient record.

11. A method for managing alarms in a sleep apnea monitor comprising of:
Monitoring Respiration (including motion) and SpO2,
Defining the clinical sequence of Apnea and LowSpO2
Assigning subalarm criteria to the adverse states in the clinical sequence;
Generating a final alarm when the patient evolves from Apnea to LowSpO2.

12. A method according to claim 11 wherein the alarm criteria are more sensitive than for Apnea and SpO2 taken separately.

13. A method for managing alarms in a sleep apnea monitor comprising of:
Monitoring Respiration (including motion), SpO2 and ST segment deviation,
Defining the clinical sequence of Apnea, LowSpO2 and ST segment deviation
Assigning subalarm criteria to the adverse states in the clinical sequence;
Generating a final alarm when the patient evolves from Apnea to LowSpO2 and to ST segment deviation.

14. A method according to claim 13 wherein the alarm criteria are more sensitive than for Apnea, SpO2 and ST segment deviation taken separately.
